(19) United States
(12) Patent Application Publication
(10) Pub. No.: US 2025/0356977 A1
(43) Pub. Date: Nov. 20, 2025

(54) METHOD AND SYSTEM FOR PREDICTING THE MEDICATION FOR AUTOIMMUNE DISEASE

(71) Applicant: NATIONAL DEFENSE MEDICAL CENTER, TAIPEI (TW)

(72) Inventors: Feng-Cheng Liu, TAIPEI (TW); Jeng-Wei Lu, TAIPEI (TW); Yi-Jung Ho, TAIPEI (TW); Ting-Yu Hsieh, TAIPEI (TW); Shan-Wen Lui, TAIPEI (TW); Ting-Chun Lin, TAIPEI (TW); Yen-Chen Chen, TAIPEI (TW); Wun-Long Jheng, TAIPEI (TW); Hsin-Ling Hsieh, TAIPEI (TW)

(21) Appl. No.: 19/202,152

(22) Filed: May 8, 2025

(30) Foreign Application Priority Data
May 17, 2024 (TW) 113118476

(51) Int. Cl. G16H 20/10; G16B 5/00; G16H 70/40

(52) U.S. Cl. CPC G16H 20/10; G16B 5/00; G16H 70/40

(57) ABSTRACT

The present invention relates to a method for predicting the medication for autoimmune disease, comprising: establishing a prediction model using a computational data through an algorithm, wherein the computational data comprises an autoimmune disease medication, a variation of a clinical index, and a proportion of a computational immune cell population.

### CROSS-REFERENCE TO RELATED APPLICATION

This application claims the benefit of Taiwan Patent Application Ser. No. 113118476, filed May 17, 2024. The disclosure of the above application is incorporated herein in its entirety by reference.

### FIELD

The present invention relates to a method for predicting the medication for autoimmune disease, especially relates to generating a prediction result of an autoimmune disease medication using a prediction model.

### BACKGROUND

The definition of an autoimmune disease comprises a symptom triggered by the immune reaction in an organism due to an immune system attacking its healthy cells, and the symptom can occur in all parts of the human body. The causes of autoimmune diseases are often difficult to identify. Aside from cases where the causes remain unknown or undiscovered and are therefore naturally indiscernible, various other factors, including environmental and genetic factors, contribute to the complexity of immune diseases, making them difficult to control or prevent. In terms of treatment, most autoimmune diseases are difficult to cure completely and are primarily managed with medication, with early and accurate control being particularly crucial.

Furthermore, many autoimmune diseases exhibit an irreversible progression. For example, in rheumatoid arthritis, in addition to swelling and pain caused by joint inflammation, prolonged inflammation over time can lead to joint stiffness, structural deformities, or functional impairments. As previously mentioned, the affected joints include the wrists, fingers, ankles, and even the cervical spine, further imposing long-term difficulties and limitations on patients.

The use of the medication for autoimmune diseases involves considerations of treatment phases and suitability, in other words, proper administration of medication with precise mechanisms of action at the appropriate time can significantly increase the likelihood of effective treatment. However, these medications are sometimes difficult to implement in the early stages of the disease and are usually introduced gradually as the disease progresses until the most appropriate medication for the patient is determined. This stepwise adjustment approach requires time, and the prolonged disease course may lead to irreversible and unnecessary burdens. At present, there remains a lack of an effective predictive method or model that could provide information to assist physicians in making more informed decisions regarding medication selection for patients.

### SUMMARY

To solve the aforementioned problem, a purpose of the present invention is to provide a method for predicting the medication for autoimmune disease, comprising: step (A): establishing a prediction model using a plurality of computational data through an algorithm, wherein each of the plurality of the computational data comprises an autoimmune disease medication, a variation of a clinical index, and a proportion of a computational immune cell population; and step (B): entering a proportion of a diagnostic immune cell population from a diagnostic data into the prediction model, thereby obtaining a prediction result of the autoimmune disease medication.

In some preferred embodiments, the algorithm comprises: a Pearson correlation analysis, a Spearman rank correlation analysis, a principal component analysis, a multiple linear regression analysis, a min-max scaling, a ROC curve analysis, a Mann-Whitney U-test, a Kruskal Wallis test, or any combination thereof.

In some preferred embodiments, the autoimmune disease medication comprises: symptom-relieving medication, immunomodulator, immunosuppressant, biologic, or any combination thereof.

In some preferred embodiments, the autoimmune disease medication comprises: a conventional synthetic disease modifying anti-rheumatic drug, (csDMARD), a biologic disease modifying anti-rheumatic drug (bDMARD), a targeted synthetic disease modifying anti-rheumatic drug (tsDMARD), or any combination thereof, wherein the tsDMARD comprises: Janus kinase inhibitor.

In some preferred embodiments, the clinical index comprises: disease activity score by 28 joints (DAS28), erythrocyte sedimentation rate (ESR), rheumatoid factor (RF), anti-cyclic citrullinated peptide antibody (anti-CCP), C-reactive protein (CRP), or any combination thereof.

In some preferred embodiments, step (A) further comprises: obtaining the proportion of the computational immune cell population by an immunophenotyping.

In some preferred embodiments, the immunophenotyping is performed by using a marker.

In some preferred embodiments, the marker comprises: CD95, CD366, HLA-DR, CD62L, CD127, CD8, KLRG-1, CD3, CD4, CD45RA, CCR7, PD-1, CD27, CD28, CD25, FOXP3, CD39, CD19, IgM, IgD, CD38, CD21, or any combination thereof.

In some preferred embodiments, the computational immune cell population comprises: T cell, B cell, basophil, neutrophil, eosinophil, dendritic cell, macrophage, natural killer cell, or any subset thereof.

The present invention further provides a system for predicting the medication for autoimmune disease, comprising a storage unit, configured to store a plurality of computational data; an input unit, configured to provide a proportion of a diagnostic immune cell population from a diagnostic data; a processing unit, connected to the storage unit for receiving the plurality of computational data and to the input unit for receiving the proportion of the diagnostic immune cell population, the process unit is configured to execute the method for predicting the medication for autoimmune disease as previously described; and an output unit, connected to the processing unit to receive and present the prediction result of the autoimmune disease medication.

In summary, through the method for predicting the medication for autoimmune disease of this invention, a patient's future medication regimen can be presented. That is, physicians can utilize this predictive method to obtain a reference for selecting the appropriate medication for the patient, thereby improving the efficiency of medical decision-making. More importantly, this method offers the potential to significantly shorten the disease course and alleviate the severity of symptoms, making it an indispensable medical tool for patients

### BRIEF DESCRIPTION OF THE DRAWINGS

FIG. 1 is a schematic diagram of the autoimmune disease medication prediction method.

FIG. 2 is a first flow chart illustrating the establishment of the autoimmune disease medication prediction model.

FIG. 3 is a flow chart illustrating the data collection step (S1).

FIG. 4 is a first flow chart illustrating the data calculation step (S2).

FIG. 5 is a second flow chart illustrating the data calculation step (S2).

FIG. 6 is a second flow chart illustrating the establishment of the autoimmune disease medication prediction model.

FIG. 7 is a schematic diagram of the autoimmune disease medication prediction system.

FIG. 8 is a chart of the regression analysis between the predictive index and the variation of the disease activity score by 28 joints (ΔDAS28).

FIG. 9 is a first comparison chart of the predictive index and the treatment result.

FIG. 10 is a first ROC curve analysis chart.

FIG. 11 is a chart of the regression analysis between the predictive index and the variation of disease activity score by 28 joints-erythrocyte sedimentation rate (ΔDAS28-ESR).

FIG. 12 is a second comparison chart of the predictive index and the treatment result.

FIG. 13 is a second ROC curve analysis chart.

### DETAILED DESCRIPTION

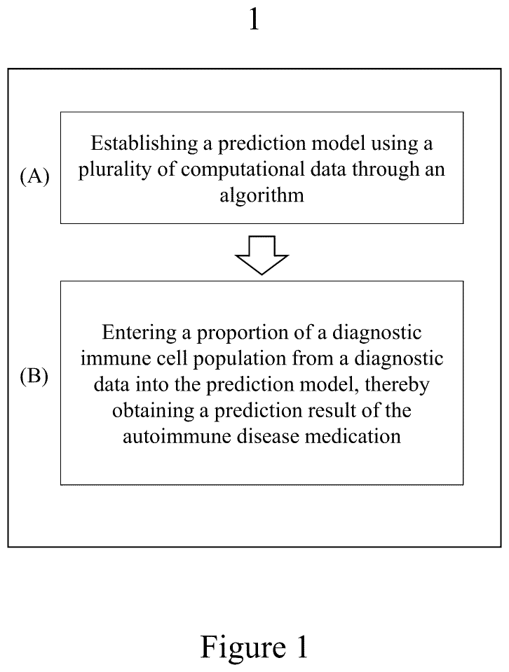

With reference to FIG. 1, which is a schematic diagram of the autoimmune disease medication prediction method 1, the present invention provides a method for predicting the medication for autoimmune disease, comprising: step (A): establishing a prediction model using a plurality of computational data through an algorithm, wherein each of the plurality of computational data comprises an autoimmune disease medication, a variation of a clinical index, and a proportion of a computational immune cell population; and step (B): entering a proportion of a diagnostic immune cell population from a diagnostic data into the prediction model, thereby obtaining a prediction result of the autoimmune disease medication.

In some preferred embodiments, the algorithm comprises: a Pearson correlation analysis, a Spearman rank correlation analysis, a principal component analysis, a multiple linear regression analysis, a min-max scaling, a ROC curve analysis, a Mann-Whitney U-test, a Kruskal Wallis test, or any combination thereof.

In some preferred embodiments, the autoimmune disease medication comprises: symptom-relieving medication, immunomodulator, immunosuppressant, biologic, or any combination thereof, concretely, comprises: non-steroidal anti-inflammatory drugs (NSAIDs), steroid, conventional synthetic disease modifying anti-rheumatic drugs (csDMARDs), Janus kinase inhibitor (JAKi), B-lymphocyte antigen inhibitor or cluster of differentiation 20 inhibitor, CD28 antagonist, interleukin-6 (IL-6) antagonist, or tumor necrosis factor alpha (TNF-α) antagonist; It is understandable that the autoimmune disease medication is used for improving or maintaining an autoimmune disease symptom of a subject, and is not limited to this. Preferably, a pharmacological pathway of the autoimmune disease medication comprises an immune pathway.

In some preferred embodiments, the clinical index comprises: 28-joint disease activity score (DAS28), ACR response criteria (ACR20/50/70), clinical disease activity index (CDAI), or simplified disease activity index (SDAI). It can be understood that the clinical index further comprises, for example, erythrocyte sedimentation rate (ESR), rheumatoid factor (RF), anti-cyclic citrullinated peptide antibody (anti-CCP), C-reactive protein (CRP), or any combination thereof.

In some preferred embodiments, step (A) further comprises: using an immunophenotyping to obtain the proportion of the computational immune cell population, wherein the proportion of the computational immune cell population can be represented as a ratio with a numerator and a denominator, and the numerator is the number of the computational immune cell population, the denominator is the number of a total immune cell population comprising the computational immune cell population.

In some preferred embodiments, the immunophenotyping is performed by using a marker, specifically, the proportion of the computational immune cell population is obtained through performing the immunophenotyping on the total immune cell population using the marker.

In some preferred embodiments, the marker comprises: CD95, CD366, HLA-DR, CD62L, CD127, CD8, KLRG-1, CD3, CD4, CD45RA, CCR7, PD-1, CD27, CD28, CD25, FOXP3, CD39, CD19, IgM, IgD, CD38, CD21, or any combination thereof, and is not limited to these.

In some preferred embodiments, the computational immune cell population comprises: T cell, B cell, basophil, neutrophil, eosinophil, dendritic cell, macrophage, natural killer cell, or any subset thereof. Clearly, the computational immune cell population is involved in the pharmacological pathway of the medication and is not limited to these.

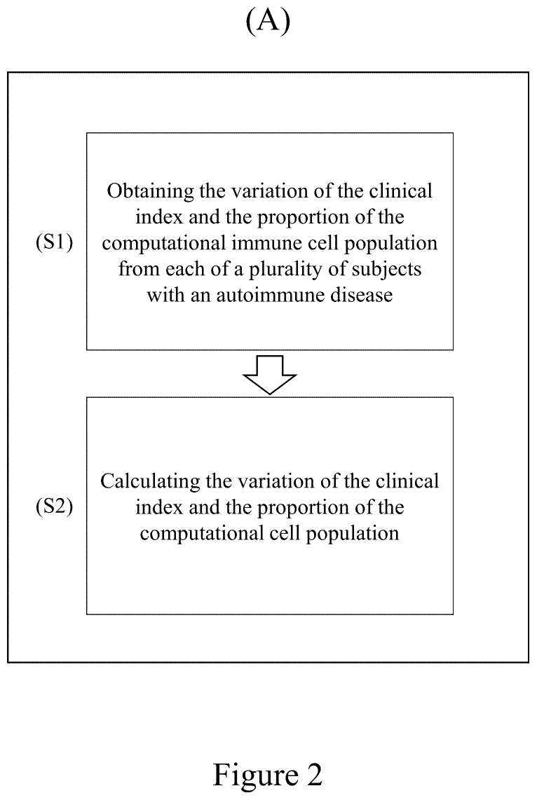

With reference to FIG. 2, which is a first flow chart illustrating the establishment of the autoimmune disease medication prediction model. With regard to the step (A), in order to establish the prediction model, the plurality of the computational data is obtained through a data collection step (S1), and the algorithm is performed in a data calculation step (S2). The data collection step (S1): obtaining the variation of the clinical index and the proportion of the computational immune cell population from each of a plurality of subjects with an autoimmune disease, wherein each of the plurality of subjects is treated with a medication for the autoimmune disease. Further, with regard to the treatment, the administration of the medication is started at a first time point and is ended at a second time point, wherein a first clinical index and the proportion of the computational immune cell population are obtained from each of the plurality of subjects at the first time point, a second clinical index is obtained from each of the plurality of subjects at the second time point, and the variation of the clinical index refers to the difference between the first clinical index and the second clinical index.

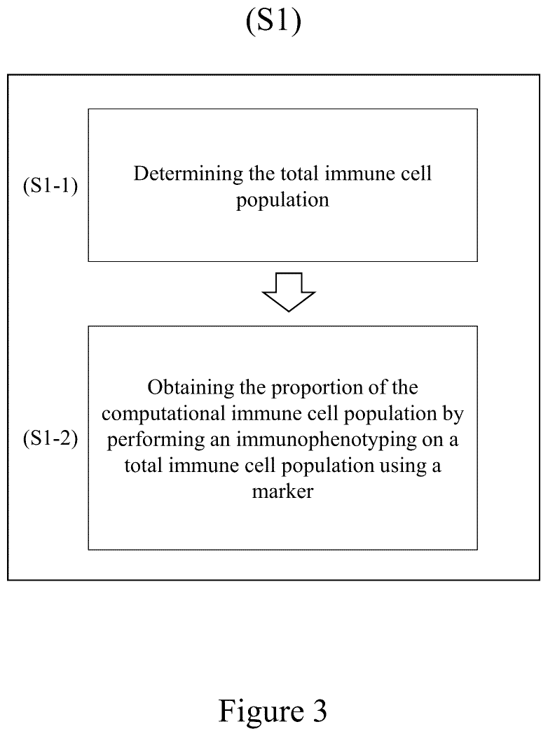

With reference to FIG. 3, which is a flow chart illustrating the data collection step (S1), in some preferred embodiments, the data collection step (S1) further comprises a data collection sub-step (S1-1): determining the total immune cell population comprising at least one of the computational immune cell population, wherein the computational immune cell population is selected from the group consisting of: T cell, B cell, basophil, neutrophil, eosinophil, dendritic cell, macrophage, natural killer cell, and any subpopulation thereof. Conceivably, the total immune cell population can be determined or decided in accordance with an immune pathway through which the medication must pass to exert its effect. For example, if a medication for an autoimmune disease is expected to exert its effect through an immune pathway involving the participation of T cells and B cells, the T cells or the B cells can be referred to as the computational immune cell population respectively, while the T cells and the B cells together can be referred to as the total immune cell population. In addition, the total immune cell population or the computational immune cell population is obtained from a sample derived from each of the plurality of the subjects, wherein the sample comprises a blood, a sweat, a spinal fluid, a saliva, a tissue fluid, or any combination thereof, apparently, the sample is not limited to these as long as it contains the computational immune cell population.

With reference to FIG. 3, which is a flow chart illustrating the data collection step (S1), in some preferred embodiments, the data collection step (S1) further comprises a data collection sub-step (S1-2): obtaining the proportion of the computational immune cell population by performing an immunophenotyping on a total immune cell population using a marker, wherein the proportion of the computational immune cell population is a ratio with a numerator and a denominator, the numerator is the number of the computational immune cell population, the denominator is the number of the total immune cell population comprising the computational immune cell population, the ratio comprises percentage or thousandths and is not limited to these. In some preferred embodiments, the marker comprises: CD95, CD366, HLA-DR, CD62L, CD127, CD8, KLRG-1, CD3, CD4, CD45RA, CCR7, PD-1, CD27, CD28, CD25, FOXP3, CD39, CD19, IgM, IgD, CD38, or CD21. Understandably, the computational immune cell population has N or N+N of the marker if N is defined as a positive integer greater than zero, the marker is chosen based on the total immune cell population, and the immunophenotyping can be performed by a flow cytometry.

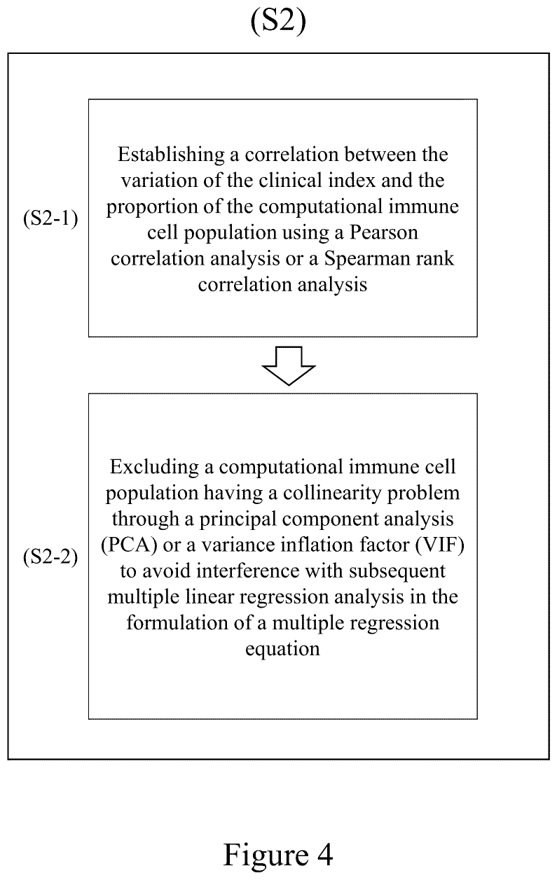

As shown in FIG. 4, FIG. 4 is first flow chart illustrating the data calculation step (S2) comprising: calculating the variation of the clinical index and the proportion of the computational cell population. Specifically, the data calculation step (S2) comprises: a data calculation sub-step (S2-1) and a data calculation sub-step (S2-2).

In some preferred embodiments, the data calculation sub-step (S2-1) comprises: establishing a correlation between the variation of the clinical index and the proportion of the computational immune cell population using a Pearson correlation analysis or a Spearman rank correlation analysis, wherein the correlation comprises: no correlation, negative correlation, or positive correlation, and a computational immune cell population having no correlation with the variation of the clinical index will be excluded in the following analysis.

In some preferred embodiments, the data calculation sub-step (S2-2) comprises: excluding a computational immune cell population having a collinearity problem through a principal component analysis (PCA) or a variance inflation factor (VIF) to avoid interference with subsequent multiple linear regression analysis in the formulation of a multiple regression equation.

In some preferred embodiments, in the multiple regression equation, the independent variable of the multiple regression equation comprises a proportion of a model immune cell population, the dependent variable of the multiple regression equation comprises a predictive index, wherein the model immune cell population is decided from the computational immune cell population through the data calculation sub-step (S2-1) and the data calculation sub-step (S2-2).

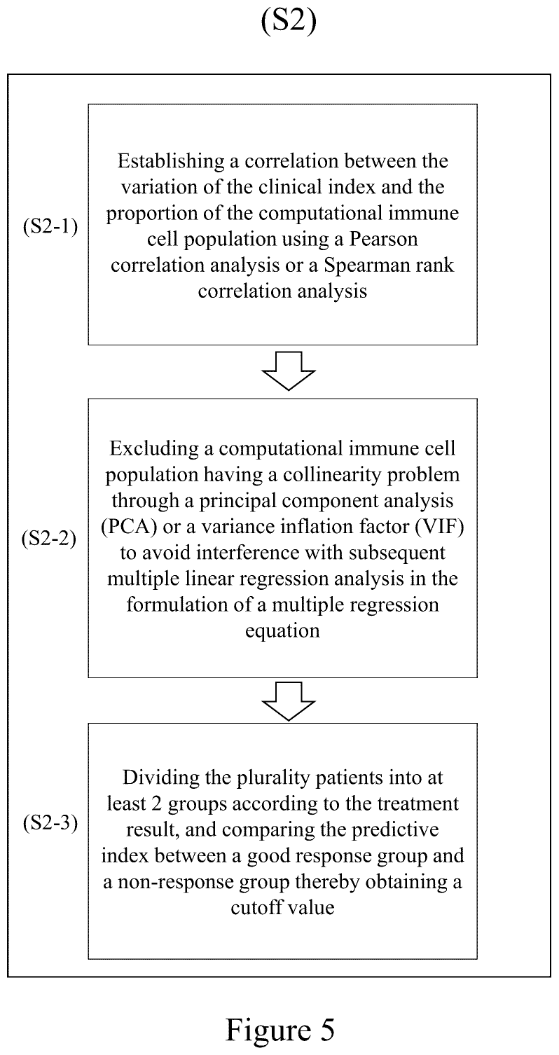

As shown in FIG. 5, which is a second flow chart illustrating the data calculation step (S2), in some preferred embodiments, the data calculation step (S2) further comprises a data calculation sub-step (S2-3) followed by the data calculation sub-step (S2-2): comparing the predictive index between a good response group and a non-response group thereby obtaining a cutoff value, wherein the good response group and the non-response group are the treatment result of the plurality of subjects received the medication for the autoimmune disease, and the cutoff value serves as the dividing line that separates the good response group from the non-response group.

In some preferred embodiments, the data calculation step (S2) further comprises using a Min-Max scaling.

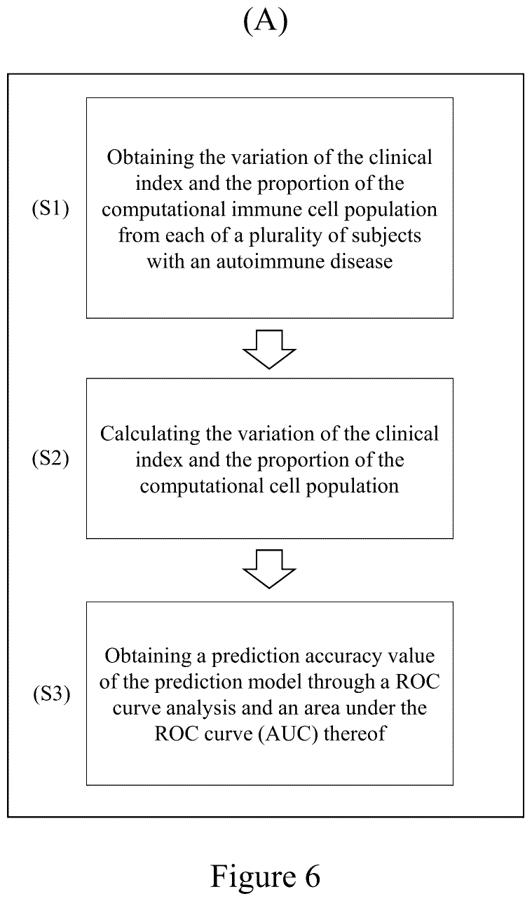

As shown in FIG. 6, which is a second flow chart illustrating the establishment of the autoimmune disease medication prediction model, in some preferred embodiments, the algorithm is further included in a data validation step (S3) followed by the data calculation step (S2) comprising: obtaining a prediction accuracy value of the prediction model through a ROC curve analysis and an area under the ROC curve (AUC) thereof.

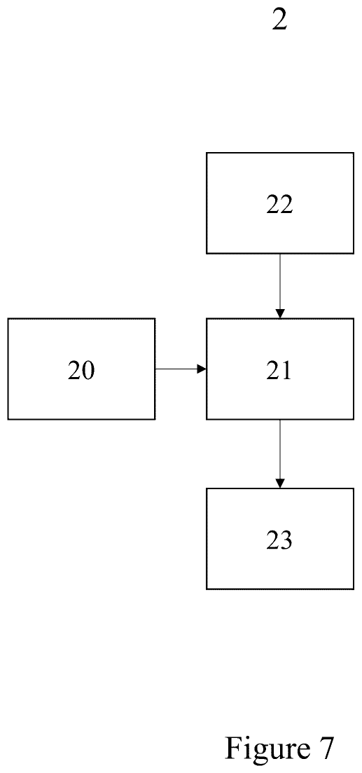

As shown in FIG. 7, which is a schematic diagram of the autoimmune disease medication prediction system 2, comprising: a storage unit 20, configured to store a plurality of computational data; an input unit 22, configured to provide a proportion of a diagnostic immune cell population from a diagnostic data; a processing unit 21, connected to the storage unit 20 thereby receiving the plurality of computational data and connected to the input unit 22 thereby receiving the proportion of the diagnostic immune cell population to perform the method for predicting the medication for autoimmune disease 1; and an output unit 23, connected to the processing unit 21 to receive and present the prediction result of the autoimmune disease medication.

In some preferred embodiments, the processing unit 21 can be used to process the computational data derived from the storage unit 20 and to establish the prediction model; the processing unit 21 can be used to process the diagnostic immune cell population of the diagnostic data derived from the input unit 22; the processing unit 21 can be used to obtain the prediction result of the autoimmune disease medication using the diagnostic immune cell population through the prediction model, wherein the prediction result of the autoimmune disease medication can be further obtained and presented by the output unit 23 from the processing unit 21.

In some preferred embodiments, the processing unit 21 obtains the computational data and the diagnostic data from the storage unit 20 and the input unit 22 respectively, establishes the prediction model thereby obtaining the prediction result of the autoimmune disease medication, and provides the prediction result of the autoimmune disease medication to the output unit 23, allowing the output unit 23 to present the same.

The following provides a first embodiments of the present invention, including a method for predicting the medication for autoimmune disease, comprising: a step (A): establishing a prediction model using a plurality of computational data through an algorithm, wherein each of the plurality of computational data comprises an autoimmune disease medication, a variation of a clinical index, and a proportion of a computational immune cell population; and a step (B): entering a proportion of a diagnostic immune cell population from a diagnostic data into the prediction model, thereby obtaining a prediction result of the autoimmune disease medication. With regard to the step (A), the plurality of computational data is obtained through a data collection step (S1), and the algorithm is included in a data calculation step (S2), therefore the prediction model can be established.

The data collection step (S1): obtaining the variation of the disease activity score by 28 joints and a proportion of a computational immune cell population from 13 patients with rheumatoid arthritis, specifically, the 13 patients are administered with a csDMARD, the administration is started at a first time point and is ended at a second time point, the first time point is 6 months prior to the second time point; further, a first disease activity score by 28 joints and the proportion of the computational immune cell population is obtained from each of the 13 patients at the first time point, a second disease activity score by 28 joints is obtained from each of the 13 patients at the second time point, and the difference between the first disease activity score by 28 joints and the second disease activity score by 28 joints is the variation of the disease activity score by 28 joints.

The data collection step (S1) further comprises a data collection sub-step (S1-1): determining a total immune cell population comprising at least one of a computational immune cell population, the computational immune cell population is: T cell, B cell, or any subset thereof, herein, the total immune cell population is T cell and B cell, the T cell and the B cell are derived from a blood of each of 13 patients, respectively.

The data collection step (S1) further comprises a data collection sub-step (S1-2): obtaining the computational immune cell population through an immunophenotyping on a total immune cell population by using a maker, and the proportion of the computational immune cell population is represented as percentage in which the numerator is the number of computational immune cell population comprising the T cell, the B cell, or any subset thereof, and the denominator is the number of the T cell and the B cell; wherein the marker comprises: CD95, CD366, HLA-DR, CD62L, CD127, CD8, KLRG-1, CD3, CD4, CD45RA, CCR7, PD-1, CD27, CD28, CD25, FOXP3, CD39, CD19, IgM, IgD, CD38, or CD21; Understandably, the computational immune cell population has N or N+N of the marker if N is defined as a positive integer greater than zero, and the immunophenotyping is performed by a flow cytometry.

The data calculation step (S2) sequentially includes data calculation sub-step (S2-1), (S2-2), and (S2-3).

The data calculation sub-step (S2-1): establishing a correlation between the variation of the disease activity score by 28 joints and the proportion of the computational immune cell population using a Pearson correlation analysis or a Spearman rank correlation analysis, wherein the correlation comprises: no correlation, negative correlation, or positive correlation, and the computational immune cell population having no correlation with the variation of the clinical index will be excluded in the following analysis.

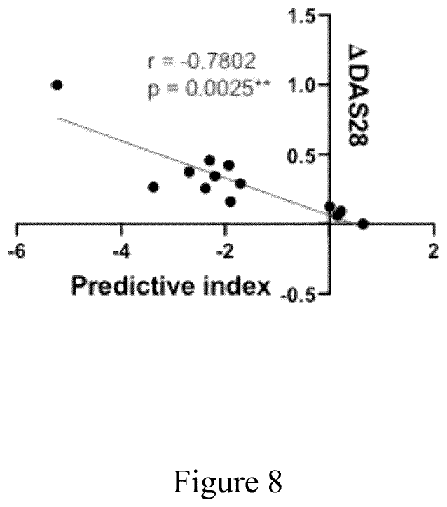

The data calculation sub-step (S2-2): excluding a computational immune cell population having a collinearity problem through a principal component analysis (PCA) or a variance inflation factor (VIF) to avoid interference with subsequent multiple linear regression analysis in the formulation of a multiple regression equation, wherein the independent variable of the multiple regression equation comprises a proportion of a model immune cell population comprising: a proportion of a regulatory T cell (Treg; CD25^{high}, CD127^{low/-}), a proportion of a type 1 regulatory T cell (Tr1; CD25⁻, Foxp3⁻, CD45RA⁻), and a proportion of a regulatory B cell (Breg; CD24^{high}, CD38^{high}), the dependent variable of the multiple regression equation is a predictive index; specifically, the model immune cell population is decided from the computational immune cell population through the data calculation sub-step (S2-1) and this data calculation sub-step (S2-2). As shown in FIG. 8, FIG. 8 is a chart of the regression analysis between the predictive index and the variation of the disease activity score by 28 joints (ΔDAS28), wherein the correlation coefficient (r) between the predictive index and the variation of the disease activity score by 28 joints (ΔDAS28) is −0.7802, and the value of significant difference (p-value) is 0.0025.

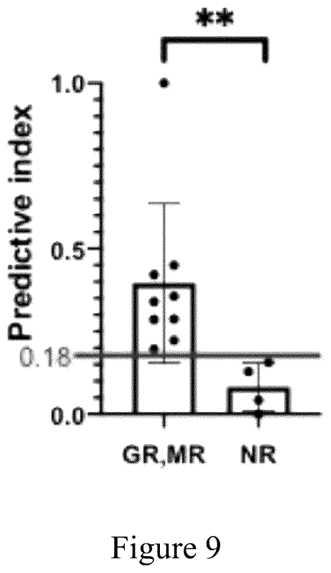

The data calculation sub-step (S2-3): dividing the plurality patients into at least 2 groups according to the treatment result, for example, a good response group (GR and MR), and a non-response group (NR), as shown in FIG. 9, which is a first comparison chart of the predictive index and the treatment result; and comparing the predictive index of the good response group and the predictive index of the non-response group to obtain a cutoff value, wherein the cutoff value serves as the dividing line between the good response group and the non-response group, and the cutoff value is 0.18, in other words, the predictive index of the patients in the good response group are larger than 0.18, the predictive index of the patients in the non-response group are lesser than 0.18.

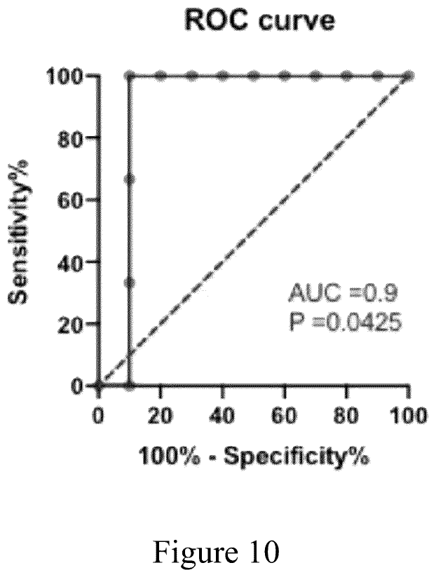

In some preferred embodiments, the algorithm is included in a data validation step (S3) followed by the data calculation step (S2): assessing a prediction accuracy value of the prediction model through a ROC curve analysis and an area under the ROC curve (AUC) thereof; as shown in FIG. 10, FIG. 10 is a first ROC curve analysis chart, the AUC is 0.9, the value of significant difference (p-value) is 0.0425, and the prediction accuracy value is 92.86%.

In some preferred embodiments, the clinical index is changed, in order words, the disease activity score by 28 joints (DAS28) is replaced by: erythrocyte sedimentation rate (ESR), rheumatoid factor (RF), anti-cyclic citrullinated peptide antibodies (anti-CCP), or C-reactive protein (CRP), the replacement can be tested by a Spearman rank correlation analysis and a multiple linear regression analysis using the model immune cell population comprising the regulatory T cell (Treg), the type 1 regulatory T cell (Tr1), and the regulatory B cell (Breg) aforementioned. It is understood that, the variation of the erythrocyte sedimentation rate (ESR), the variation of rheumatoid factor (RF), the variation of anti-cyclic citrullinated peptide antibodies (anti-CCP), or the variation of C-reactive protein (CRP) correlates with the proportion of the regulatory T cell (Treg), the proportion of the type 1 regulatory T cell (Tr1), and the proportion of the regulatory B cell (Breg), and the value of significant difference (p-value) thereof is less than 0.05, as shown in Table 1.

TABLE 1

| | Computational | | |
|---|---|---|---|
| Variation of the | immune cell | Correlation | |
| clinical index (Δ) | population | coefficient (r) | p-value |
| ΔDAS28 | Treg | −0.7758 | 0.0017 |
| | Tr1 | −0.8330 | 0.0004 |
| | Breg | −0.6352 | 0.0171 |
| ΔCRP | Treg | −0.7626 | 0.0022 |
| | Tr1 | −0.6396 | 0.0161 |
| | Breg | −0.4794 | 0.0624 |
| ΔESR | Treg | −0.6807 | 0.0090 |
| | Tr1 | −0.6519 | 0.0135 |
| | Breg | −0.6007 | 0.0156 |

The following provides a second embodiment of the present invention, including a method for predicting the medication for autoimmune disease, comprising: a step (A): establishing a prediction model using a plurality of computational data through an algorithm, wherein each of the plurality of computational data comprises an autoimmune disease medication, a variation of a clinical index, and a proportion of a computational immune cell population; and a step (B): entering a proportion of a diagnostic immune cell population from a diagnostic data into the prediction model, thereby obtaining a prediction result of the autoimmune disease medication. With regard to the step (A), the plurality of computational data is obtained through a data collection step (S1), and the algorithm is included in a data calculation step (S2), therefore the prediction model can be established.

The data collection step (S1): obtaining the variation of the DAS28-ESR and a proportion of a computational immune cell population from 16 patients with rheumatoid arthritis, specifically, the 16 patients are administered with a Janus kinase inhibitor (JAKi), the administration is started at a first time point and is ended at a second time point, the first time point is 6 months prior to the second time point; further, a first DAS28-ESR and the proportion of the computational immune cell population is obtained from each of the 16 patients at the first time point, a second DAS28-ESR is obtained from each of the 16 patients at the second time point, and the difference between the first DAS28-ESR and the second DAS28-ESR is the variation of the DAS28-ESR (percentage change).

The data collection step (S1) further comprises a data collection sub-step (S1-1): determining a total immune cell population comprising at least one of a computational immune cell population, the computational immune cell population is: T cell, B cell, or any subset thereof, herein, the total immune cell population is T cell and B cell, the T cell and the B cell are derived from a blood of each of 16 patients, respectively.

The data collection step (S1) further comprises a data collection sub-step (S1-2): obtaining the computational immune cell population through an immunophenotyping on a total immune cell population by using a maker, and the proportion of the computational immune cell population is represented as percentage in which the numerator is the number of computational immune cell population comprising the T cell, the B cell, or any subset thereof, and the denominator is the number of the T cell and the B cell; wherein the marker comprises: CD95, CD366, HLA-DR, CD62L, CD127, CD8, KLRG-1, CD3, CD4, CD45RA, CCR7, PD-1, CD27, CD28, CD25, FOXP3, CD39, CD19, IgM, IgD, CD38, or CD21; understandably, the computational immune cell population has N or N+N of the marker if N is defined as a positive integer greater than zero, and the immunophenotyping is performed by a flow cytometry.

The data calculation step (S2) sequentially includes data calculation sub-step (S2-1), and (S2-2).

The data calculation sub-step (S2-1): establishing a correlation between the variation of the DAS28-ESR and the proportion of the computational immune cell population using a Pearson correlation analysis or a Spearman rank correlation analysis, wherein the correlation comprises: no correlation, negative correlation, or positive correlation, and the computational immune cell population having no correlation with the variation of the clinical index will be excluded in the following analysis. As shown in Table 2, it is demonstrated that the proportion of 14 of the T cell or its subset and the proportion of 20 of the B cell or its subset positively or negatively correlates with the variation of DAS28-ESR (ΔDAS28-ESR).

TABLE 2

| | | ΔDAS28-ESR | | | | |
|---|---|---|---|---|---|---|
| Proportion of the | | | | | Spearman rank | |
| computational immune | | Pearson correlation analysis | | | correlation analysis | |
| cell population | | r | R² | p-value | r | p-value |
| T | TEM | 0.5310 | 0.2819 | 0.0343* | 0.4000 | 0.1259 |
| | EM T Fas⁺ | 0.4771 | 0.2276 | 0.0617 | 0.5235 | 0.0397* |
| Th | Naïve Th | −0.4872 | 0.2373 | 0.0556 | −0.5618 | 0.0257* |
| | Th CD28⁺CD27⁺ | −0.5164 | 0.2667 | 0.0406* | −0.4550 | 0.0779 |
| | Th CD28⁻CD27⁻ | 0.5125 | 0.2627 | 0.0424* | 0.2794 | 0.2937 |
| | Th CD57⁺ | 0.6041 | 0.3649 | 0.0132* | 0.4412 | 0.0889 |
| | CD39⁺Helios⁻ | 0.5349 | 0.2861 | 0.0328* | 0.3824 | 0.1447 |
| | Treg | | | | | |
| Tc | CM Tc | 0.6318 | 0.3992 | 0.0087** | 0.5147 | 0.0436* |
| | CM Tc Fas⁺ | 0.6302 | 0.3971 | 0.0089** | 0.5618 | 0.0257* |
| | Tc PD-1⁺ | 0.5840 | 0.3410 | 0.0175* | 0.6824 | 0.0046** |
| | Tc CD57⁺ | 0.7887 | 0.6221 | 0.0003*** | 0.7059 | 0.0030** |
| | Tc HLADR⁺ | 0.5146 | 0.2648 | 0.0414* | 0.4324 | 0.0961 |
| | Tc CD28⁺CD27⁻ | 0.5009 | 0.2509 | 0.0481* | 0.2588 | 0.3319 |
| | Tc CD28⁻CD27⁻ | 0.7195 | 0.5176 | 0.0017** | 0.5735 | 0.0223* |
| B | B | −0.7004 | 0.4905 | 0.0025** | −0.6765 | 0.0051** |
| | B CD21⁺ | −0.6946 | 0.4825 | 0.0028** | −0.6588 | 0.0068** |
| | B HLADR⁺ | −0.6501 | 0.4226 | 0.0064** | −0.6265 | 0.0110* |
| | Marginal B | −0.6838 | 0.4676 | 0.0035** | −0.6206 | 0.0120* |
| | Marginal B | −0.6641 | 0.4411 | 0.0050** | −0.6206 | 0.0120* |
| | CD21⁺ | | | | | |
| | Marginal B | −0.6179 | 0.3818 | 0.0107* | −0.5824 | 0.0200* |
| | HLADR⁺ | | | | | |
| | Marginal B PD-1⁺ | −0.5437 | 0.2956 | 0.0295* | −0.4059 | 0.1201 |
| | Naïve B | −0.5664 | 0.3208 | 0.0222* | −0.4588 | 0.0758 |
| | Naïve B CD21⁺ | −0.5604 | 0.3141 | 0.0239* | −0.4412 | 0.0889 |
| | Naïve B HLADR⁺ | −0.5515 | 0.3041 | 0.0268* | −0.4706 | 0.0679 |
| | SM B | −0.4735 | 0.2242 | 0.0640 | −0.6500 | 0.0078** |
| | SM B CD21⁺ | −0.5226 | 0.2731 | 0.0378* | −0.6324 | 0.0101* |
| | SM B HLADR⁺ | −0.5136 | 0.2638 | 0.0419* | −0.5441 | 0.0316* |
| | DN B | −0.6347 | 0.4029 | 0.0083** | −0.5912 | 0.0178* |
| | DN B CD21⁺ | −0.6918 | 0.4785 | 0.0030** | −0.6853 | 0.0044** |
| | DN B HLADR⁺ | −0.5375 | 0.2889 | 0.0318* | −0.5500 | 0.0295* |
| | B | −0.6290 | 0.3957 | 0.0090** | −0.6088 | 0.0141* |
| | CD38^{dim}CD21^{dim} | | | | | |
| | Class non-switched | −0.5259 | 0.2766 | 0.0364* | −0.6441 | 0.0085** |
| | memory B | | | | | |
| | B IgM⁺ | −0.6172 | 0.3809 | 0.0109* | −0.4618 | 0.0738 |
| | B IgM⁺ CD27⁻ | −0.5402 | 0.2919 | 0.0307* | −0.4206 | 0.1062 |

The proportion of the computational immune cell population in the Table 2, as mentioned above, is obtained at the first time point when the treatment with JAKi started, the variation of the DAS28-ESR is the change between the DAS28-ESR obtained at first time point and the DAS28-ESR obtained at the second time point (the end of the JAKi treatment). With reference to the table 2, (r) value is the correlation coefficient, the correlation between the variation of the DAS28-ESR and the proportion of the computational immune cell population will be negative if the (r) value is negative, and vice versa; R² is coefficient of determination; significance differences are represented by p-values: * for p<0.05, ** for p<0.01, and *** for p<0.001.

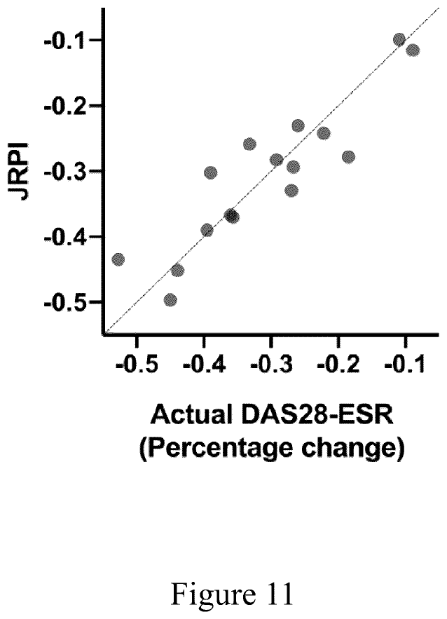

The data calculation sub-step (S2-2): excluding a computational immune cell population having a collinearity problem through a principal component analysis (PCA) or a variance inflation factor (VIF) to avoid interference with subsequent multiple linear regression analysis in the formulation of a multiple regression equation, wherein the multiple regression equation is Y=(0.0541×A)+(−0.3656×B)+(−0.0511×C)+(0.01308×D)+(0.005809×E)−0.4541, wherein the independent variable of the multiple regression equation comprises a proportion of a model immune cell population comprising: (A): CM Tc cell, (B): CD38^{dim}, CD21^{dim} B cell, (C): class non-switched memory B cell, (D): EM T Fas⁺ cell and (E): Naïve Th cell, the dependent variable of the multiple regression equation (Y) is the predictive index; specifically, the model immune cell population is decided from the computational immune cell population through the data calculation sub-step (S2-1) and this data calculation sub-step (S2-2). As shown in FIG. 11, FIG. 11 is a chart of the regression analysis between the predictive index and the variation of disease activity score by 28 joints-erythrocyte sedimentation rate (ΔDAS28-ESR), in the FIG. 11, the predictive index is represented as JRPI (JAKi treatment response predictive index), the JRPI is correlated with the variation of the DAS28-ESR, the coefficient of determination R² is 0.8218.

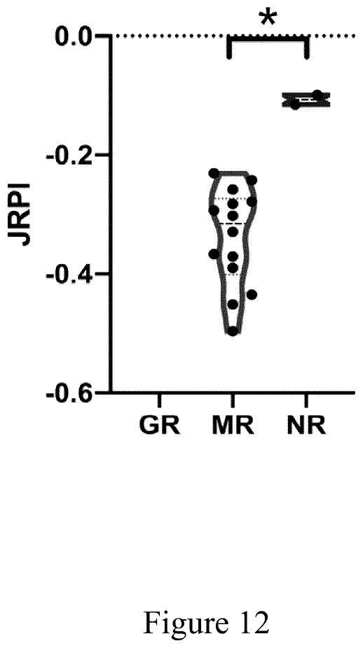

As shown in FIG. 12, which is a second comparison chart of the predictive index and the treatment result. The plurality patients are divided into at least 2 groups according to the treatment result, for example, a good response group (GR), a moderate response group (MR) and a non-response group (NR); further, the predictive index of the good response group, the moderate response group, and the non-response group are compared through Mann-Whitney U test, therefore a significant difference (p-value) is 0.0167. Clearly, the predictive index can accurately distinguish the treatment results observed clinically.

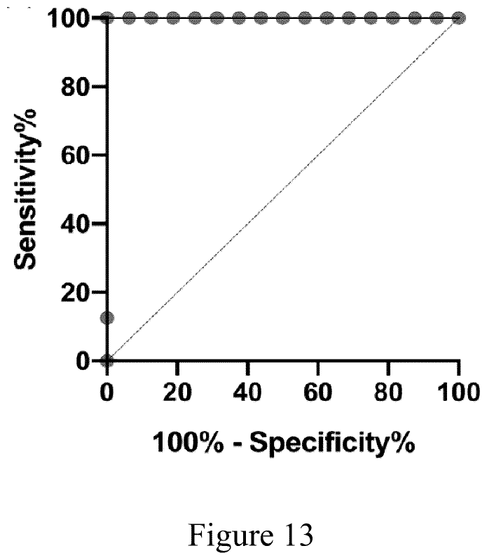

In some preferred embodiments, the algorithm is further included in a data validation step (S3) followed by the data calculation step (S2): assessing a prediction accuracy value of the prediction model through a ROC curve analysis and an area under the ROC curve (AUC) thereof; as shown in FIG. 13, the AUC is 1, and the prediction accuracy value is 100.00%.

Accordingly, the correlation between the treatment result and the predictive index is not limited to various relative relationship, for example, the predictive index of the good response group can be higher or lower than the predictive index of the non-response group, due to the difference of the medication for autoimmune disease, the model immune cell population, and the multiple regression equation thereof.

The foregoing description merely illustrates the preferred embodiments of the present invention and should not be construed as limiting the scope of the invention. Any simple equivalent changes or modifications made in accordance with the scope of the patent claims and the description of the invention shall fall within the scope covered by the patent of the present invention.

What is claimed is:

1. A method for predicting the medication for autoimmune disease, comprising:
step (A): establishing a prediction model using a computational data through an algorithm, wherein the computational data comprises an autoimmune disease medication, a variation of a clinical index, and a proportion of a computational immune cell population; and
step (B): entering a proportion of a diagnostic immune cell population from a diagnostic data into the prediction model, thereby obtaining a prediction result of the autoimmune disease medication.

2. The method as claimed in claim 1, wherein the algorithm comprises: a Pearson correlation analysis, a Spearman rank correlation analysis, a principal component analysis, a multiple linear regression analysis, a min-max scaling, a ROC curve analysis, a Mann-Whitney U-test, a Kruskal Wallis test, or any combination thereof.

3. The method as claimed in claim 1, wherein the autoimmune disease medication comprises:
symptom-relieving medication, immunomodulator, immunosuppressant, biologic, or any combination thereof.

4. The method as claimed in claim 1, wherein the autoimmune disease medication comprises:
a conventional synthetic disease modifying anti-rheumatic drug, (csDMARD), a biologic disease modifying anti-rheumatic drug (bDMARD), a targeted synthetic disease modifying anti-rheumatic drug (tsDMARD), or any combination thereof.

5. The method as claimed in claim 4, wherein the tsDMARD comprises: Janus kinase inhibitor (JAKi).

6. The method as claimed in claim 1, wherein the clinical index comprises: disease activity score by 28 joints (DAS28), erythrocyte sedimentation rate (ESR), rheumatoid factor (RF), anti-cyclic citrullinated peptide antibody (anti-CCP), C-reactive protein (CRP), or any combination thereof.

7. The method as claimed in claim 1, wherein the step (A) further comprises: obtaining the proportion of the computational immune cell population of a sample by an immunophenotyping using a marker.

8. The method as claimed in claim 7, wherein the immunophenotyping is performed using a flow cytometry.

9. The method as claimed in claim 7, wherein the sample comprises a blood, a sweat, a spinal fluid, a saliva, tissue fluid, or any combination thereof.

10. The method as claimed in claim 7, wherein the marker comprises: CD95, CD366, HLA-DR, CD62L, CD127, CD8, KLRG-1, CD3, CD4, CD45RA, CCR7, PD-1, CD27, CD28, CD25, FOXP3, CD39, CD19, IgM, IgD, CD38, CD21, or any combination thereof.

11. The method as claimed in claim 1, wherein the computational immune cell population comprises: T cell, B cell, basophil, neutrophil, eosinophil, dendritic cell, macrophage, natural killer cell, or any subset thereof.

12. The method as claimed in claim 1, wherein the step (A) further comprises:
obtaining a first immune cell population from the computational immune cell population through a correlation analysis using the variation of the clinical index;
obtaining a model immune cell population from the first immune cell population through a principal component analysis; and
obtaining a multiple regression equation through a multiple linear regression analysis using the model immune cell population, thereby establishing the prediction model.

13. The method as claimed in claim 12, wherein a dependent variable of the multiple regression equation comprises a predictive index.

14. The method as claimed in claim 12, wherein an independent variable of the multiple regression equation comprises a proportion of the model immune cell population.

15. The method as claimed in claim 13, wherein a cutoff value is used to establish the prediction model, and the step (A) further comprises:
deciding the cutoff value based on the comparison of the predictive index and a clinical result of the administration of the autoimmune disease medication.

16. The method as claimed in claim 15, wherein the prediction result of the autoimmune disease medication is determined by the cutoff value.

17. A system for predicting the medication for autoimmune disease, comprising: a processing unit, configured to receive a proportion of a diagnostic immune cell population from a diagnostic data, and to generate a prediction result of an autoimmune disease medication using a prediction model.

18. The system as claimed in claim 17, wherein the processing unit is further configured to receive a computational data, and to establish the prediction model by processing the computational data using an algorithm comprising: a correlation analysis, a principal component analysis, a multiple linear regression analysis, a min-max scaling, a ROC curve analysis, a Mann-Whitney U-test, a Kruskal Wallis test, or any combination thereof.

19. The system as claimed in claim 18, wherein the computational data comprises the autoimmune disease medication, a variation of a clinical index, and a proportion of a computational immune cell population.

20. The system as claimed in claim 18, further comprising:
an input unit, connected to the processing unit and configured to provide the diagnostic data;
an output unit, connected to the processing unit and configured to present the prediction result of the autoimmune disease medication; and
a storage unit, connected to the processing unit and configured to provide the computational data.
